(19) United States
(12) Patent Application Publication
(10) Pub. No.: US 2012/0248140 A1
(43) Pub. Date: Oct. 4, 2012

(54) PORTABLE HAND SANITATION DISPENSER

(76) Inventors: Mert Iseri, Evanston, IL (US); Yuri Malina, Evanston, IL (US)

(21) Appl. No.: 13/438,653

(22) Filed: Apr. 3, 2012

Related U.S. Application Data
(63) Continuation-in-part of application No. 12/945,343, filed on Nov. 12, 2010.
(60) Provisional application No. 61/297,346, filed on Jan. 22, 2010.

(51) Int. Cl. B65D 35/28

(52) U.S. Cl. 222/1; 222/101

(57) ABSTRACT

This disclosure is directed to a portable hand sanitation solution dispenser that may include a dispenser portion, a solution reservoir and an attachment assembly. The dispenser portion may include a housing, a hand roller having a cam portion, an electronic module and a pump assembly. The cam portion is moved for activating and deactivating the pump assembly. The reservoir may be configured as a replaceable cartridge with an opening through which the pump dip tube extends into the solution reservoir. The hand roller may be disposed at the bottom end of the dispenser so that all the dispensed solution is on the hand of the user after swiping the hand roller.

This application is a continuation in part that that claims the benefit of and priority to U.S. application Ser. No. 12/945,343 filed on Nov. 12, 2010 titled “Wearable Sanitizing Gel Dispenser Kit and Associated Methods,” that claims benefit of and priority to U.S. Provisional Application No. 61/297,346 filed on Jan. 22, 2010 titled “Wearable Gel Dispenser,” all of which are incorporated herein by reference in their respective entireties.

### BACKGROUND

The embodiments herein generally relate to a personal hygiene and sanitation device and particularly relate to a portable hand sanitation device. The embodiments herein more particularly relate to a portable hand sanitation gel dispenser for health care workers in hospitals for dispensing a specified amount of gel.

A hand sanitizer is preferably used in addition to a hand washing performed with soap and water. A plurality of sanitizer materials currently available in the market include gel, foam and aqueous solutions or liquids. A group of active ingredients used in the sanitizer materials may include isopropanol, ethanol, n-propanol, or povidone-iodine. Further, a group of inactive ingredients may also be used in the sanitizer materials. The group of inactive ingredients may include polyacrylic acid for alcohol gels, humectants such as glycerin for liquid rubs, propylene glycol and essential oils of plants. An alcohol based hand sanitizer is more effective in killing the germs than soaps.

One conventional device is a wall mounted hand sanitizer that includes a sensor to detect a presence of the hands underneath the dispenser. The sensor activates the dispenser to spray an alcohol based sanitizing gel. Since this hand sanitizer is mounted on the wall at a plurality of selected locations, the users are able to access to the sanitizers only at the selected locations.

Another conventional device is a portable hand sanitizer that may be attached to the user's clothing. The hand sanitizer includes a push button that is connected to a nozzle of the dispenser. The user applies an appropriate force on the push button to activate a pump to spray a sanitizer gel from a dispenser nozzle until the pressing of the push button is stopped. When the pressing of the push button is stopped, the pump is deactivated. Since this portable hand sanitizer does not control the amount of sanitizer solution discharged from the nozzle, the user is not aware of the remaining amount of gel present in the hand sanitizer reservoir.

In view of the foregoing, there is a need for a portable hand sanitation solution dispenser to enhance portability and to provide a secure attachment mechanism. Also, there is a need for a portable hand sanitation solution dispenser to increase a usage frequency of the sanitizer gel. Further, there is a need for a hand sanitation solution dispenser that provides a behaviorally appropriate dispensing activation and mechanism.

The above identified shortcomings, disadvantages and problems of conventional sanitizer dispensers are addressed herein and overcome by this disclosure directed to a portable hand sanitation gel dispenser that may include a behaviorally appropriate dispensing mechanism, that may be connected to a plurality of places on a clothing easily and securely, that dispenses a metered dose or a constant amount of a sanitation gel at each time of use, that includes a refillable cartridge of the sanitation gel for refilling the sanitation gel easily and efficiently, and that avoids dripping of the sanitation solution when the dispenser is not in use.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The disclosure will be more readily understood in view of the following description of the preferred embodiments and the accompanying drawings in which like reference numerals represent like elements and wherein:

FIG. 1A illustrates a perspective view of the portable hand sanitation solution dispenser, according to an embodiment herein.

FIG. 1B illustrates a side sectional view of the portable hand sanitation solution dispenser of FIG. 1A, according to an embodiment herein.

FIG. 1C illustrates a rear perspective view of the portable hand sanitation solution dispenser of FIG. 1A, according to an embodiment herein.

FIG. 1D illustrates a side elevation view of the portable hand sanitation solution dispenser of FIG. 1A, according to an embodiment herein.

FIG. 2A illustrates a front elevation view of a dispenser portion and a front elevation view of a refillable cartridge compartment in a portable hand sanitation solution dispenser in an unassembled condition, according to an embodiment herein.

FIG. 2B illustrates an exploded perspective view of the portable hand sanitation solution dispenser, according to an embodiment herein.

FIG. 2C illustrates an exploded rear perspective view of the portable hand sanitation solution dispenser, according to an embodiment herein.

FIG. 3 illustrates a perspective view of a portable hand sanitation solution dispenser, according to an embodiment herein.

The specific features of the embodiments herein are shown in some drawings and not in others, which is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced are shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments described herein are directed to a portable hand sanitation solution dispenser that may include a dispenser portion, a sanitizer solution reservoir and an attachment assembly.

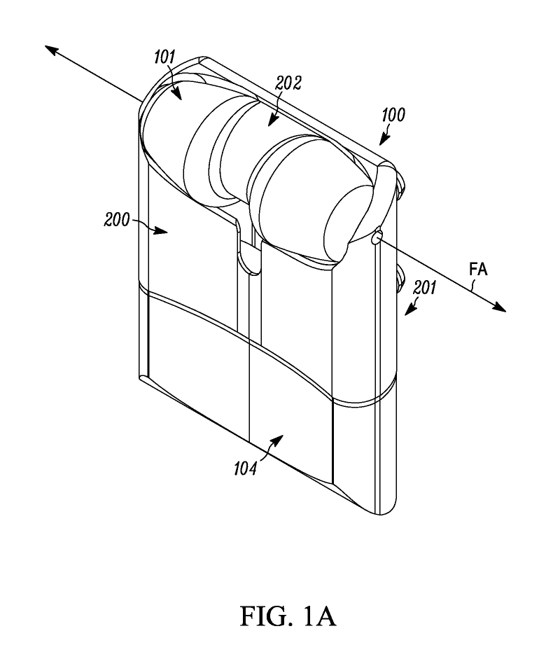

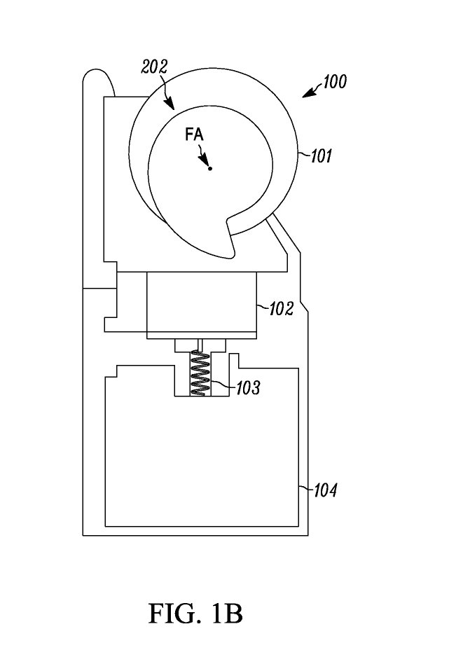

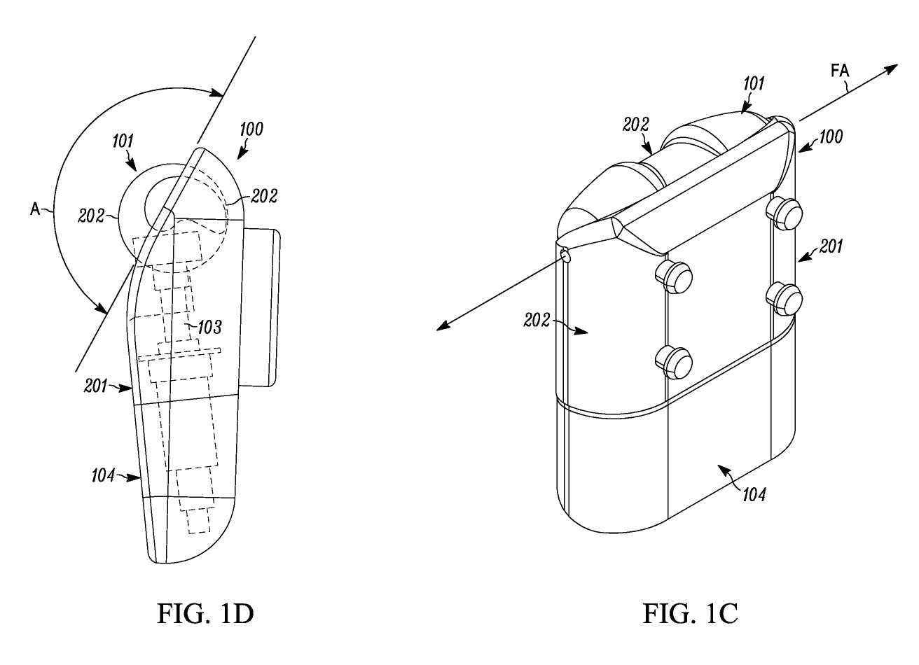

According to one embodiment herein, the dispenser portion may include a housing, a hand roller having a cam portion, an electronic module and a pump assembly. The housing may include a dispenser opening including a hand roller opening (that extends about a perimeter of the roller to expose a substantial portion of the roller, i.e. greater than approximately 135 degrees and preferably greater than approximately 180 degrees, angle A in FIG. 1D), and a pump discharge opening (that extends from the roller opening toward the reservoir and generally centrally located), and a pump dip tube opening. The housing may also include a concave front face or a generally curved configuration. The hand roller may be configured as a generally ellipsoidal component with varying diameters, a prolate spheroid, rounded, back-to-back frustum of right circular cone, obround or any other similar or like configuration to provide the intended functionality. The hand roller also includes the cam portion defined therein generally having a recessed area (for the pump body at rest, i.e., hand roller not being actuated) and a curved ramp having an increasing radius from a low part to adjacent the outer surface of the remainder of the hand roller. The cam portion activates and deactivates the pump assembly. The pump assembly may include a conventional hand pump body having a head with an outlet or discharge nozzle, and a suction line or dip tube that extends into and is disposed within the reservoir. The solution reservoir is defined by a housing for the sanitizer solution and is generally configured as a replaceable cartridge. The reservoir housing may include an opening (with a temporary removable or transferable seal for storage before use) through which the pump dip tube extends into the solution reservoir and a pair of connection tabs that releasably engage the swiping compartment.

According to an embodiment herein, the solution reservoir may further include two one-way valve cartridges. The two one-way valve cartridges hold a sanitation solution within the compartment, but permit quick refilling of the reservoir.

According to an embodiment herein, the hand roller is rotatably connected to housing at a fixed axis. The hand roller rotates about the fixed axis when a tangential force is applied to the outer engagement portions. As a result of the rotation of the hand roller, the cam portion rotates to engage and depress the head of the pump in the downward direction.

According to an embodiment herein, the downward motion of the head activates the pump in order to draw a quantity of sanitizer solution from the sanitizer solution reservoir.

According to an embodiment herein, the amount of sanitizer solution drawn by the pump is proportional to a linear downward displacement of the head by the cam portion. Since the downward displacement of the cam portion is fixed, the amount of sanitizer solution withdrawn from the reservoir is also a preset or constant amount on a single use.

According to an embodiment herein, the predetermined specific amount of sanitizer solution that is withdrawn from the reservoir is a metered dose for a single use.

According to an embodiment herein, the pump nozzle sprays the sanitizer solution from the reservoir onto the hand of a user that actuated the hand roller which is properly oriented and positioned as a result thereof.

According to an embodiment herein, the electronic module comprises a plurality of counters, sensors and alarm unit.

According to an embodiment herein, the user may be a healthcare worker in a medical centre or any other concerned with personal hygiene.

According to an embodiment herein, the portable hand sanitation gel dispenser may be connected to any one of a plurality of places on clothing by an attachment assembly such as, for example only, pins that project from the housing 200 for engagement with a clip 400 to 402 that has locking apertures formed therein to define a releasable engagement. According to an embodiment herein, the attachment assembly may include a plurality of interlocking pins. According to an embodiment herein, the attachment assembly may include a magnetic sticker board and a recipient board. The magnetic sticker board attaches to the recipient board. According to an embodiment herein, the magnetic sticker board further may include a plurality of tiny magnets. The recipient board also may include a plurality of tiny magnets. According to an embodiment herein, the polarity of the magnets provided in the magnetic sticker board and the recipient board are opposite to each other. According to an embodiment herein, the attachment assembly is machine washable.

According to an embodiment herein, a portable hand sanitation solution dispenser comprises a hand roller disposed in a lowermost orientation or the bottom end of the dispenser. The hand roller is swiped in a downward motion to move the cam portion to activate the pump that draws the sanitizer solution from the refillable cartridge reservoir and dispenses it onto the hand of the user. The roller is configured such that all the dispensed solution is disposed on the hand of the user.

According to an embodiment, the swiping compartment housing may include two halves, parts, pieces or any other suitable configuration of components in order to achieve the intended functionality so as to define and provide an interior space to accommodate the roller with the cam, the pump, and a circuit board printed with the required electronic and powering circuits. The roller rotates in response to a swipe action of a user, after or while engaging the roller, that likewise rotates the cam portion. The cam portion in turn engages a head of the pump to activate the pump to dispense a metered amount or a constant amount of sanitation solution from the refillable cartridge. The time of dispensation and the frequency of usage are recorded by the electronic module in a standard data format or suitable other format in a data store. The remaining or residual amount of sanitizer solution in the reservoir cartridge is calculated and monitored based thereon and an alert is provided to a user when the calculated residual amount of solution in the reservoir cartridge is below a preset value indicating a low level and time for replacement. The alert may be provided through a physical, audio or a visual output. The audio output may be an alarm, the video output may be a lighting of a LED, and the physical output may be a vibration.

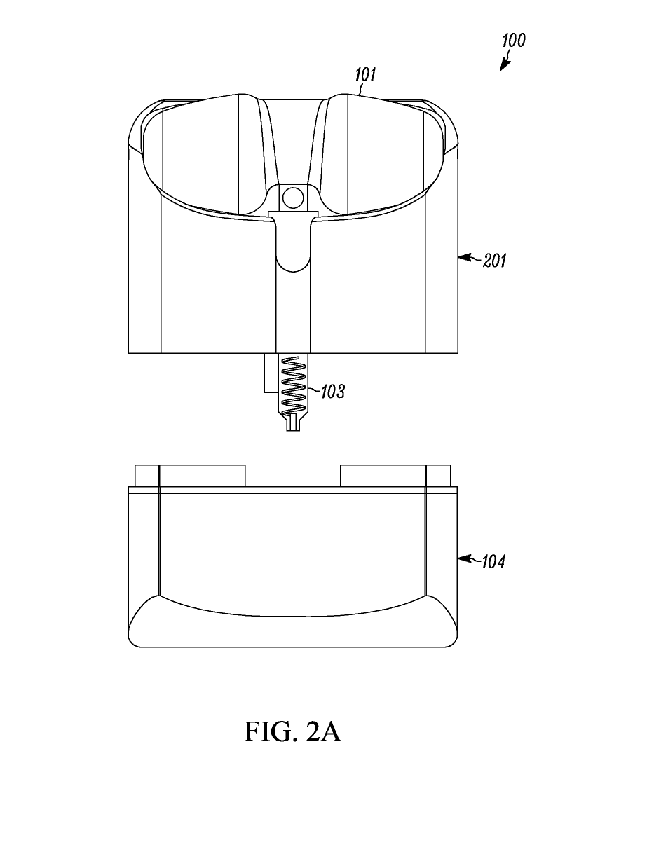

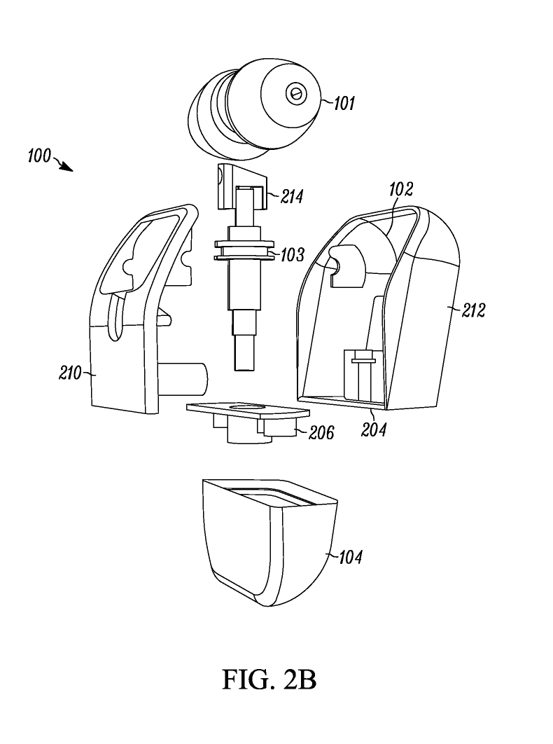

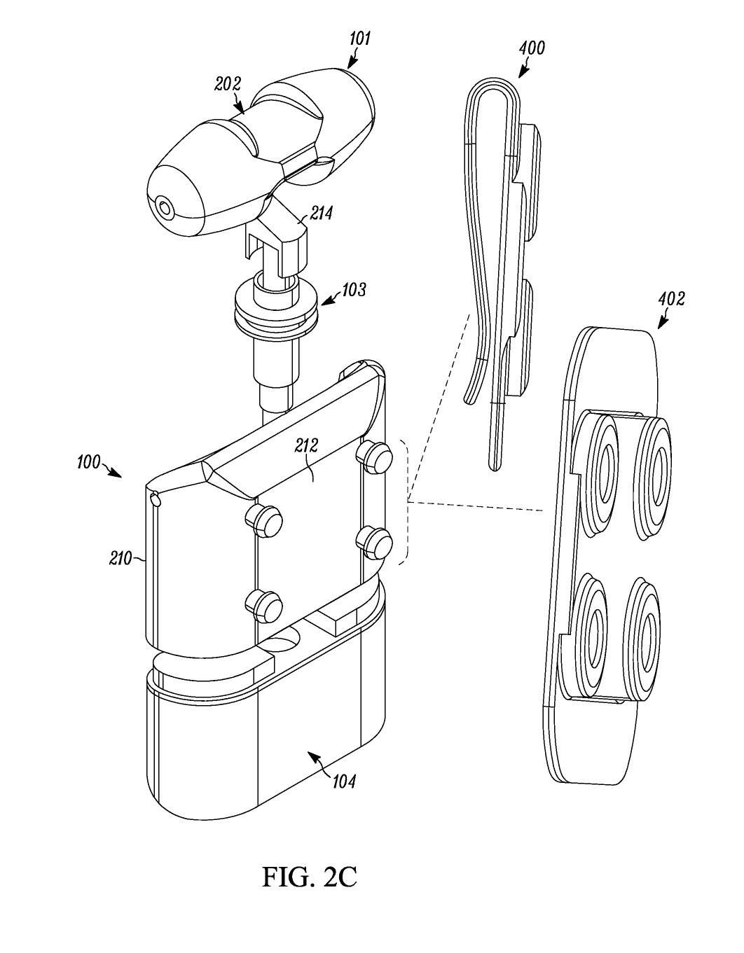

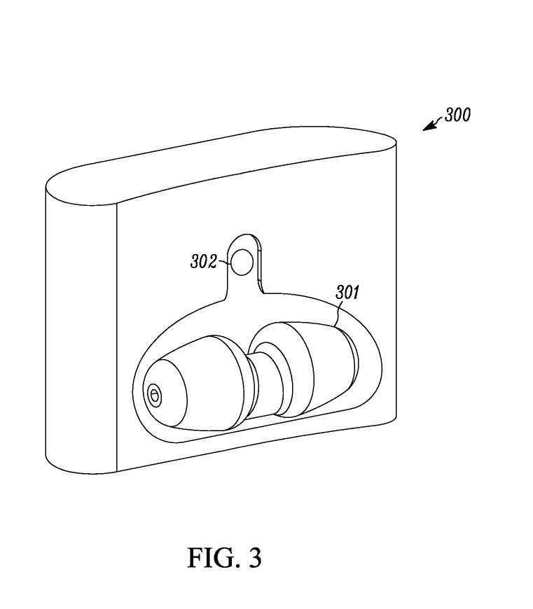

FIGS. 1A and 1C illustrate perspective views of a portable hand sanitation solution dispenser 100, according to an embodiment herein, and FIGS. 1B and 1D illustrate side sectional and side elevation views of the portable hand sanitation solution dispenser 100 of FIG. 1A. With respect to FIGS. 1A, 1B, 1C and 1D, the portable hand sanitation solution dispenser 100 may include a dispenser portion 201 including a housing 200, hand roller 101 having a cam portion 202, the electronic module 102 (FIG. 1B), the pump assembly 103 (FIG. 1B) and the solution reservoir 104. The hand roller 101 may have any suitable configuration as described herein and preferably rotates about a fixed axis FA. The cam portion 202 moves likewise with rotation of hand roller 101 to contact and slidingly engage the head of the pump assembly connected to the housing 200 at the pump slot 204 (FIG. 2B). The pump 103 is actuated by the cam portion to dispense a metered amount or a constant amount of sanitizing solution from the solution reservoir 104 on each time of use.

When the portable hand sanitation solution dispenser 100 is disposed in a first vertical orientation, the hand roller 101 is at the topmost position of the dispenser 100. The electronic module 102 is disposed below the hand roller 101. The solution reservoir 104 is disposed in the lowermost position of this embodiment of the portable hand sanitation solution dispenser 100. The pump assembly 103 is arranged in between the electronic module 102 and the solution reservoir 104. A fastening assembly is connected to a back portion of the housing 200 to secure the dispenser 100 with a clothing of the user.

FIG. 2A illustrates a front elevation, partially exploded view of the dispenser, according to an embodiment herein. With respect to FIG. 2A, the portable hand sanitation solution dispenser 100 comprises a dispenser portion 201 and a refillable cartridge reservoir 104. The dispenser portion 201 and the refillable cartridge reservoir 104 are coupled to each other through a conventional fastening structure such as, for example, tabs having enlarged distal ends extending from the reservoir 104 that protrude into the dispenser portion 201 while being biased inwardly and then engage apertures formed in the housing 200, or any other suitable connection, etc., to provide the intended functionality. The fastening structure may also include male and female connectors provided respectively in the dispenser portion 201 and the refillable cartridge compartment 104. The pump assembly 103 may project out of the dispenser portion 201 for insertion into the refillable cartridge reservoir 104 in an assembled configuration.

FIGS. 2B and 2C illustrate an exploded perspective views of the portable hand sanitation solution dispenser 100, according to an embodiment herein. With respect to FIGS. 2B and 2C, the dispenser portion 201 may include a front enclosure 210, a rear enclosure 212, a hand roller 101 having a cam portion 202, a housing for an electronic module 102 and a pump assembly 103. The pump 103 may also include a dip tube that extends into the refillable cartridge reservoir 104 through an opening. The downward motion of the pump head 214 activates the pump 103 to draw a metered dose of the sanitation solution disposed in the refillable cartridge reservoir 104.

The refillable cartridge reservoir 104 may further include two one-way valve cartridges. The two one-way valves 206 hold the sanitation solution within the reservoir 104, but facilitate quick refilling of the cartridge 104.

The front enclosure 202 and the rear enclosure 203 are connected to each other by conventional coupling structure to provide a housing 200 to accommodate the components of the dispensing compartment of the portable hand sanitizer solution dispenser 100. The front enclosure 202 has a front wall that has a concave or curved configuration so as to expose more of the roller for contact during the actuation of the roller and to avoid deposit of the sanitizer solution onto the roller rather than the hand of the user. The front and rear enclosures 202 and 203 cooperatively define the housing 200 and include a hard back top rear edge/corner/lip/etc. so as to move of the roller for the functionality as described herein.

The housing for electronic module 102 may include a mechanical counter that is connected to the pump head 214 in order to increment by one unit after each discharge of a metered amount of sanitizer solution or hand wash. Furthermore, the housing for electronic module 102 may include a sensor for determining whether a critical amount of hand sanitizer solution remains in the cartridge 104. The critical amount is the minimum amount of the hand sanitizer solution left in the cartridge 104. The sensor may be connected to an alarm to provide an audio, visual or physical alert when the critical amount of hand sanitizer solution in the cartridge 104 is below a preset value.

FIG. 3 illustrates a front elevation perspective view of a portable hand sanitation solution dispenser 100, according to an embodiment herein. With respect to FIG. 3, the hand roller 301 is disposed at the lowermost or bottom end of the portable hand sanitation solution dispenser 300. The cam portion of the hand roller 301 contacts and slidingly engages the pump head that further moves in a vertical direction downwardly. The movement of the cam portion activates the pump that is arranged above the hand roller 301 more near to the top end of the portable hand sanitation solution dispenser 300.

The hand roller 301 is substantially as otherwise described herein as is the remainder of the dispenser 300 and accordingly, will not be repeated for the sake of brevity. The pump draws a metered dose or a constant amount of the sanitation solution from the refillable cartridge reservoir. The pump further dispenses the solution through an outlet over the hands of the user. The roller is arranged in such a manner to ensure that all the gel is dispensed on the hand.

The hand sanitizer gel dispenser of the embodiments herein is portable and is fixed to the plurality of places on the clothing of the user. The portable nature of the hand sanitizer gel dispenser minimizes the need for a wall mounted gel dispenser at different locations.

The hand sanitizer gel dispenser also provides a metered dose of gel on a single use. Also the user is intimated through an alert or alarm, when the sensors detect the critical amount of gel present in the gel compartment.

The portable hand sanitation gel dispenser of the embodiments herein is cost effective and provides least complicated component assembly. Also the portable hand sanitation gel dispenser avoids the dripping of the sanitizer gel when it is not in use.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

What is claimed is:

1. A sanitizing solution dispenser comprising:
a dispenser portion and a sanitizer solution reservoir, wherein the reservoir is removably connected to the dispenser portion and includes a sanitizer solution disposed therein and an opening; the dispenser portion including: a housing; a roller rotatably connected to the housing, the roller having a cam portion; and a pump assembly connected to the housing, the pump assembly including a pump head, having a discharge nozzle, and a dip tube that extends through the opening into the reservoir; wherein a metered dose is dispensed from the discharge nozzle when the roller is actuated such that the cam portion contacts and moves the pump head in order to activate the pump assembly.

2. The dispenser of claim 1, wherein the reservoir further includes a one-way fill valve.

3. The dispenser of claim 1, wherein the housing further includes a roller opening that extends about a perimeter of the roller to expose a substantial portion of the roller.

4. The dispenser of claim 3, wherein the substantial portion is greater than approximately 135 degrees.

5. The dispenser of claim 1, wherein the housing further includes a front enclosure having a concave front wall.

6. The dispenser of claim 1, wherein the cam portion further includes a recessed area and a curved ramp such that the pump head is disposed in the recessed area when the roller is not being actuated in the sliding contact engagement with the ramp when the roller is being actuated.

7. A method for dispending sanitizing solution using a sanitizing solution dispenser to a hand of a user, the method comprising: attaching the sanitizing solution dispenser to the clothing of the user, the dispenser including: a housing; a roller rotatably connected to the housing, the roller having a cam portion; a pump assembly connected to the housing, the pump assembly including a pump head, having a discharge nozzle, and a dip tube that extends through the opening into the reservoir; a clothing attachment member connected to the dispenser portion to facilitate such attaching; and actuating the roller using a selective hand motion by the user to contact the pump head and timely activate the pump assembly to dispense a metered dose from the discharge nozzle.

8. The method of claim 7, further comprising the roller at a bottom of the dispenser.

9. The method of claim 7, wherein the housing further includes a roller opening that extends about a perimeter of the roller to expose a substantial portion of the roller.

10. The dispenser of claim 9, wherein the substantial portion is greater than approximately 135 degrees.

11. The dispenser of claim 9, wherein the housing further includes a front enclosure having a concave front wall.

12. The dispenser of claim 7, wherein the cam portion further includes a recessed area and a curved ramp such that the pump head is disposed in the recessed area when the roller is not being actuated in the sliding contact engagement with the ramp when the roller is being actuated.
